(19) United States
(12) Patent Application Publication
(10) Pub. No.: US 2012/0095542 A1
(43) Pub. Date: Apr. 19, 2012

(54) INTRALUMINAL MEDICAL DEVICE

(75) Inventor: Kurt J. Tekulve, Ellettsville, IN (US)

(73) Assignee: Cook Incorporated, Bloomington, IN (US)

(21) Appl. No.: 12/905,738

(22) Filed: Oct. 15, 2010

(51) Int. Cl. A61F 2/84; A61F 2/82

(52) U.S. Cl. 623/1.11; 623/1.2; 623/1.36; 623/1.46

(57) ABSTRACT

A multi-sided intraluminal medical device having a self-expanding frame and a pair of guide bars located on opposite ends of the frame and adapted to enable the frame to be retracted for delivery or retrieval from either end is provided. The intraluminal device may further include a partial or full covering that is circumferentially attached to the frame. Such a medical device may be used as a stent to maintain an open lumen in a vessel (e.g., a vein, artery, or duct), as a valve, or as an occlusion device.

### FIELD

This disclosure relates generally to medical devices. More specifically, this disclosure relates to intraluminal devices.

### BACKGROUND

In recent years, intraluminal devices, instruments for placement of these devices, and a variety of minimally invasive techniques used to deploy and retrieve these devices have been developed. Minimally invasive intraluminal devices, such as stents, stent grafts, occlusion devices, artificial valves, shunts, etc., have been used to successfully treat a number of conditions that before their development either lacked an adequate solution or had to be surgically treated. Coronary and peripheral stents have been proven in recent years to provide a superior means of maintaining vessel patency. In addition, these stents have been successfully used in conjunction with grafts as a repair for an abdominal aortic aneurysm, with fibers or other materials as occlusion devices, and as an intraluminal support for artificial valves, among other uses.

One of the goals associated with the design of a new stent or related device focuses on providing the device with sufficient radial strength in order to allow the device to adequately supply a force against the wall of the vessel, thereby, preventing unwanted migration of the device. An additional goal associated with peripheral use of a device, is having the device be resistant to external compression. In this regard, self-expanding stents are known to be superior to balloon expandable stents. Thus another goal is being able to design a device that can be delivered intraluminally to a target location in a vessel in as small of a configuration as possible, while still being capable of adequate expansion. This goal becomes increasingly difficult when the device also requires the use of a fabric or other covering, which requires being folded with the device for placement into a delivery catheter. The development of a basic intraluminal device having a fabric covering that is capable of being delivered with a low profile, has a sufficient expansion ratio to permit implantation in larger vessels (when desired), is capable of conforming to the shape of the vessel, and that can be delivered or retrieved from the vessel in either direction would be beneficial.

### SUMMARY

In overcoming the enumerated drawbacks and other limitations of the related art, the present disclosure provides a multi-sided intraluminal medical device having a self-expanding frame and a pair of guide bars located on opposite ends of the frame and adapted to enable the frame to be retracted for delivery or retrieval from either end.

According to one aspect of the present disclosure, the intraluminal device comprises a frame and a pair of guide bars; the frame including a plurality of side elements interconnected by a plurality of oppositely facing bends. The frame is configured to move between a collapsed state in which the bends are compressed into near proximity with the side elements for delivery and retrieval and an expanded state in which the frame expands allowing the side elements to engage the blood vessel. The pair of guide bars are configured to couple with the oppositely facing bends of the frame.

According to another aspect of the present disclosure, the intraluminal device further includes a covering attached to at least a portion of the circumference of the frame. The covering may be a full covering in which the aperture of frame is enclosed, thereby, allowing the intraluminal device when deployed into a body vessel to act as an occlusion device. Alternatively, the covering may be a partial covering having a triangular or similar shape. The partial covering allows the intraluminal device upon deployment into a body vessel to act as an artificial valve.

According to another aspect of the present disclosure, a method for delivering and retrieving the intraluminal device is provided. More specifically, the intraluminal device may be delivered and retrieved from a targeted location in a body vessel from either direction using one of the pair of guide bars. During the delivery of the intraluminal device, a pushing mechanism contacts one of the guide bars of the device to push or move the device through the catheter to the targeted location in the body vessel. During retrieval, the retrieval hook grasps one of the guide bars to pull or move the device from the targeted location in the body vessel and through the catheter.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is a side perspective view of the frame of an intraluminal device prepared according to the teachings of the present disclosure;

FIG. 2 is a cross-sectional view of the frame of the intraluminal device of FIG. 1 shown in its collapsed state within a delivery catheter;

FIG. 3 is a side perspective view of the frame of an intraluminal device prepared according to another aspect of the present disclosure shown positioned in a body lumen;

FIG. 4A is a side perspective view of an intraluminal device prepared according to one aspect of the present disclosure including the frame of FIG. 1 and a full covering;

FIG. 4B is a side perspective view of an intraluminal device prepared according to one aspect of the present disclosure including the frame of FIG. 1 and a partial covering;

FIG. 5A is a cross-sectional view of a body lumen depicting the intraluminal device of FIG. 4 deployed in a manner in which the first guide bar leads the way;

FIG. 5B is a cross-sectional view of a body lumen depicting the intraluminal device of FIG. 4 deployed in a manner in which the second guide bar leads the way;

FIG. 6A is a cross-sectional view of a body lumen depicting the deployed intraluminal device of FIG. 5B being retrieved through the use of the second guide bar; and

FIG. 6B is a cross-sectional view of a body lumen depicting the deployed intraluminal device of FIG. 5A being retrieved through the use of the first guide bar.

### DETAILED DESCRIPTION

The following description is merely exemplary in nature and is in no way intended to limit the present disclosure or its application or uses. The present disclosure specifically contemplates other embodiments not illustrated but intended to be included in the appended claims. It should be understood that throughout the description and drawings, corresponding reference numerals indicate like or corresponding parts and features.

The present disclosure generally provides a multi-sided intraluminal medical device having a self-expanding frame and a pair of guide bars located on opposite ends of the frame and adapted to enable the frame to be retracted for delivery or retrieval from either end. Optionally, the intraluminal medical device may further include a partial or full covering that is circumferentially attached to the frame. Such a medical device may be used as a stent to maintain an open lumen in a vessel (e.g., a vein, artery, or duct), a valve, or an occlusion device.

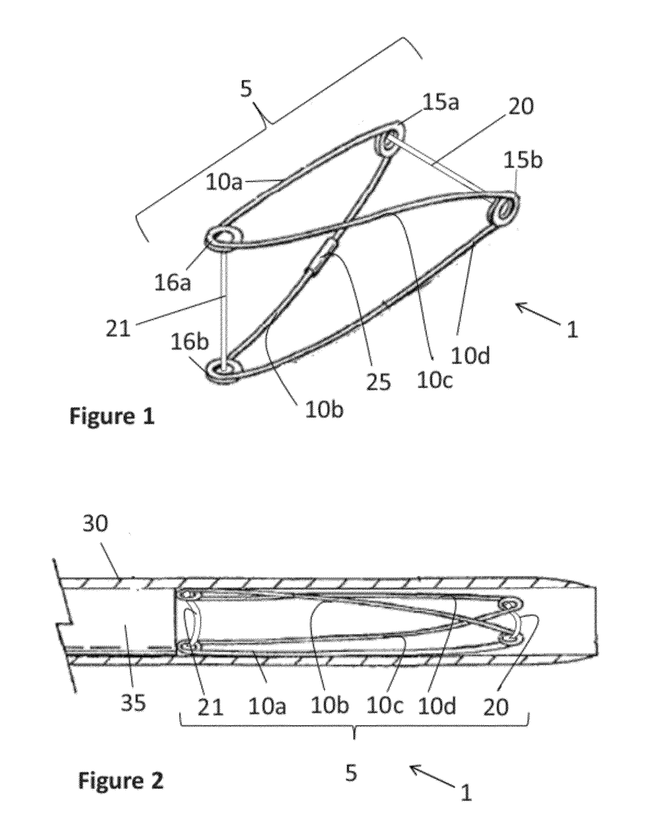

Referring to FIG. 1, the frame 5 of the intraluminal device 1 has a plurality of side elements 10 interconnected by a plurality of oppositely facing bends 15(a-b), 16(a-b). The frame 5 is configured to move between a collapsed state in which the bends 15, 16 are compressed into near proximity with the side elements 10 for delivery and retrieval and an expanded state in which the frame 5 expands allowing the side elements 10 to engage the blood vessel. The first 20 and second 21 guide bars are configured to couple with the oppositely facing bends 15, 16.

Still referring to FIG. 1 the frame 5 is preferably made from a resilient material, such as a metal wire comprised of stainless steel or a superelastic material (e.g., Nitinol). Although a wire with a round surface is depicted in the figures, one skilled-in-the-art will understand that other types of wires, e.g., flat, square, or triangular, may be used to form the frame. The frame 5 may be comprised of multiple wire components coupled together through the use of more than one attachment mechanism 25 or a single component, e.g., wire strand, the ends of which are coupled together using a single attachment mechanism 25. In both cases, the resulting device 1 exhibits a closed circumference. The single wire strand may be fabricated into the frame 5 through the use of stamping or cutting from a sheet (e.g., by laser, etc.) or via various molding techniques or a similar method. Further finishing procedures can be performed after the frame 5 has been cut or formed, including, but not limited to, polishing, deburring, and adding surface treatments or coatings. Such surface treatments or coatings may include a therapeutic agent, such as antiproliferative agents, anti-inflammatory agents, and antiplatelet agents, among others

The attachment mechanism 25 may be include a small piece of a metal cannula or tube with the ends of the frame inserted therein and secured with solder, a weld, adhesive, crimping, or the like. The ends of the frame may also be joined directly without a metal cannula through the use of soldering, welding, or any other method known to one skilled-in-the-art.

The frame 5 may have four sides 10(a-d) with each side preferably being roughly equal in length. One skilled-in-the-art will understand that the frame may be formed into any polygonal shape having sides of varying length, including but not limited to, a pentagon, hexagon, and octagon, without exceeding the scope of the disclosure. Other geometric shapes and configurations suitable for use with the intraluminal device of the present disclosure is described by Pavcnik et al. in U.S. Patent Publication No. 2009/0157169, the contents of which are hereby incorporated by reference in their entirety.

The bends 15, 16 that interconnect the sides 10 may be comprised of a simple 90° turn or a coil with approximately one and a quarter turns, among other types of bends. The coil bend will produce superior bending fatigue characteristics than that of the simple bend when the frame 5 is made from stainless steel. On the other hand, when the frame 5 is formed from Nitinol (NiTi) or any other superelastic alloy, the use of a bend may actually be preferable. Other types of bends suitable for use with the intraluminal device of the present disclosure is described by Pavcnik et al. in U.S. Pat. No. 6,508,833, the entire contents of which are hereby incorporated by reference.

The size of the wire used to construct the intraluminal device 1 is predetermined based upon the desired size of device and the intended application for the device. For example, for use as an occlusion device a wire having a thickness of about 0.254 mm (0.010″) would be selected to form a 10 mm square frame, while wire having a thickness of about 0.356 mm (0.014″) and 0.406 mm (0.016″) would be used for forming 20 mm and 30 mm square frames, respectively. If the wire selected for use in the device 1 is too stiff, the profile of the device in its collapsed state will be larger than necessary, thereby, making delivery into the targeted vessel more difficult. In addition, the profile of the device in its expanded state may not conform well to the vessel wall or even potentially damage the vessel wall.

Referring now to FIG. 2, the method of deploying the medical device 1 into a vessel involves placing the device 1 with its frame 5 in a collapsed state into a delivery device 30, such as a catheter. In the collapsed state, the adjacent sides 10(a-d) of the frame are generally beside each other in close proximity. The guide bars 20, 21 are designed such that they can bend to allow the frame 5 to move to its collapsed state. In order to advance and deploy the device from the distal end of the delivery catheter 30, a pushing mechanism 35 is placed into the lumen of the catheter 30. When the intraluminal device 1 is fully deployed within a vessel, it moves to its expanded state as depicted in FIG. 1. In the expanded state, the sides 10(a-d) of the frame, being made of resilient material, conform to the shape of the vessel wall, such that when viewed on its end, the device 1 will exhibit a circular appearance when deployed in a round vessel.

The intraluminal device 1 has a first pair of two opposite bends 15a, 15b oriented at one end of the device 1 and a second pair of opposite bends 16a, 16b oriented at the other end of the device 1. The first pair of bends 15a, 15b are rotated approximately 90° with respect to the second pair of bends 16a, 16b when viewed in cross-section. The bending stresses introduced into the frame by the bends 15, 16, will apply a force radially outward against the wall of a vessel to hold the device 1 in place and prevent vessel closure. The guide bars 20, 21 are located such that they coupled to the opposite bends in different pairs of bends. More specifically, one guide bar 20 is coupled to the first pair of opposite bends 15a, 15b, while the second guide bar 21 is coupled to the second pair of opposite bends 16a, 16b.

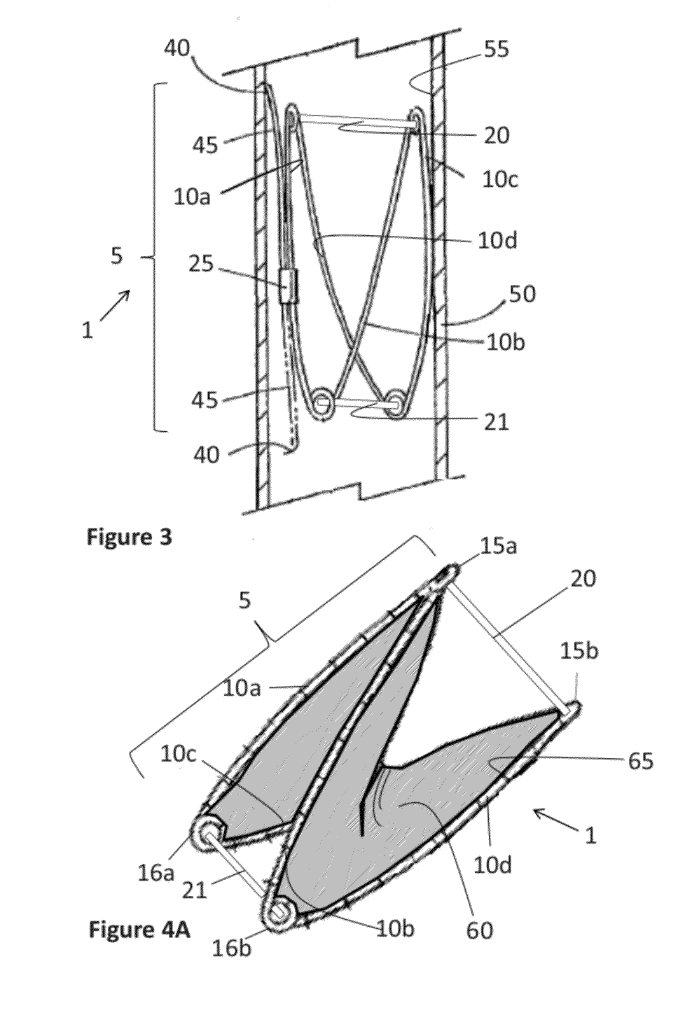

Referring now to FIG. 3, according to another aspect of the present disclosure, at least one of the sides may include one or more barbs 40 to anchor the device 1 to the wall 55 of a vessel or lumen 50 following deployment. The barb 40 may be attached to the end of a wire, a strut, or any other structure 45 attached to the frame 5 and so configured as to be able to anchor the device 1 within the lumen 50. The structure 45 may be coupled to the frame through an existing connection point 25 or by any other means. The structure 45 may be formed as an extension of one of the sides 10 of the frame 5 beyond the closed circumference of the frame 5. In order to facilitate anchoring, the barb 40 end of the structure may comprise a bend, hook, or a portion of the structure that is ground to a sharpened point for better penetration of the wall 55 of the lumen 50.

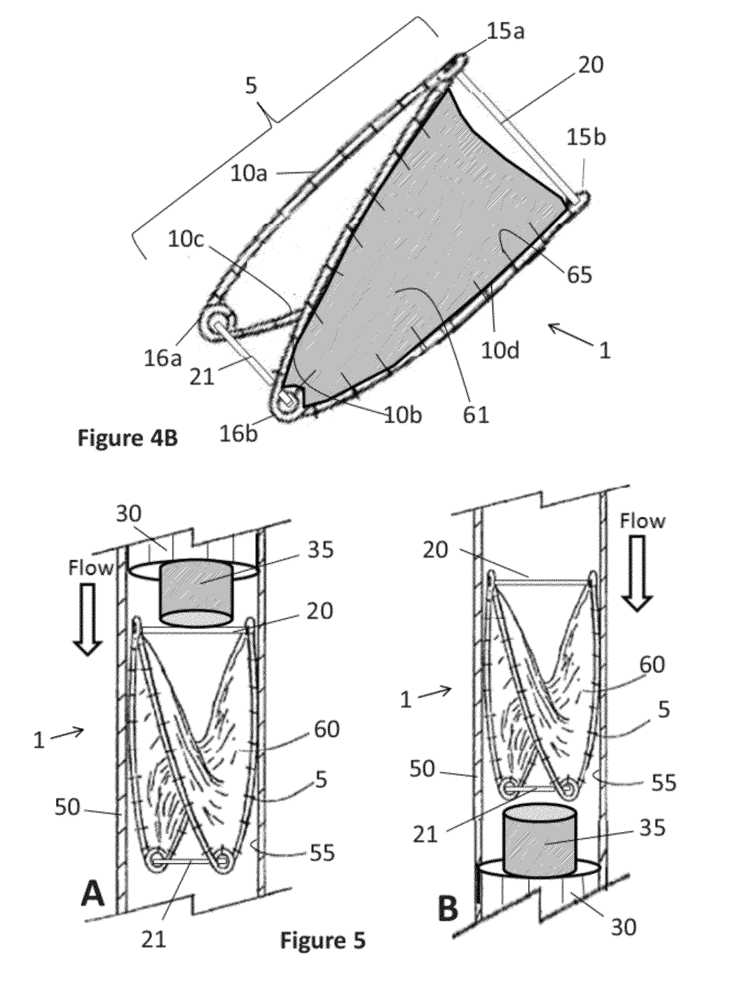

Referring now to FIGS. 4A and 4B, the intraluminal device 1 of the present disclosure may optionally include a partial 61 or full covering 60 attached to at least a portion of the circumference of the frame 5. The covering 60, 61 may be comprised of a sheet of fabric, collagen (such as small intestinal submucosa), or any other flexible material known to one skilled-in-the-art. The cover 60, 61 may be attached to the frame 5 by means of sutures 65, adhesive, heat sealing, “weaving” together, cross-linking, or other known means.

A full covering 60 will generally cover the entire aperture of the frame 5 as shown in FIG. 4A. When the device 1 having a full covering 60 is deployed and allowed to move to its expanded state, it may be used as an occlusion device to block a duct or vessel, close a shunt, repair a defect, or other application where complete or substantially complete prevention of flow of fluid through the body lumen or vessel is necessary or desired. The design of the intraluminal device 1 of the present disclosure permits it to be used successfully in large vessels such as the aorta. Preferably, the intraluminal device 1 when used as an occlusion device should have side 10 lengths that are at least about 50% or larger than the vessel diameter in which it is to be deployed.

A partial covering 61, will generally be triangular in shape as shown in FIG. 4B. The partial covering 61 will extend over approximately half of the aperture of the frame 5 of the intraluminal device 1. When the intraluminal device 1 having such a partial covering 61 is deployed in the lumen of a vessel and allowed to move to its expanded state, the device 1 can act as an artificial valve, such as the type used to correct valvular incompetence. In this application, the partial covering 61 may be displaced toward the wall of the vessel due to positive fluid pressure or flow through the vessel in one direction, e.g., normal blood flow, thereby maintaining a passageway through the frame 5 and the lumen of the vessel. As the muscles relax, thereby, producing flow in the opposite direction, e.g., retrograde blood flow, the partial covering 61 will act as a normal valve by catching the backward flowing blood and closing the lumen of the vessel. One skilled-in-the-art will understand that in addition to the triangular covering, other possible configurations of the partial covering 61 that result in the cupping or trapping of fluid in one direction may be used.

Referring once again to FIGS. 1 and 2, the guide bars 20, 21 coupled the oppositely spaced bends 15(a,b), 16(a,b) in the intraluminal device may be comprised of the same or different material than the sides 10(a-d) in the frame 5 of the intraluminal device 1. The guide bars 20, 21 are adapted to allow the guide bars 20, 21 to flex or bend in order for the intraluminal device 1 to move from the expanded state to the collapsed state. Preferably, the guide bars are comprised of a superelastic material (i.e., Nitinol) or a flexible polymer matrix, including, but not limited to, polyesters, polytetrafluoroethylene, expanded polytetrafluoroethylene, and nylon. The guide bars 20, 21 may be attached to the sides 10(a-d) of the frame 5 through the use of welding, soldering, adhesives, or any other means of fastening or attachment known to one skilled-in-the-art. One example of mechanically attaching the guide bars 20, 21 to the frame 5 is to form the guide bars 20, 21 into a dumbbell shape such that both ends of the guide bar are larger than the rest of the bar and the opening in the coil bend 15, 16 through which the bar 20, 21 is placed.

According to another aspect of the present disclosure a method for using the previously described intraluminal device 1 in a body vessel. This method generally includes both delivering and retrieving the intraluminal device 1. Referring now to FIGS. 5A and 5B, delivering the intraluminal device 1 to a targeted location in the body includes, first, introducing a delivery catheter 30 having a proximal and distal end into the body vessel 50. The distal end of the delivery catheter 30 is positioned to be proximate to a targeted location in the body vessel 50. Then an intraluminal device 1 having a frame 5 and guide bars 20, 21 (with a partial or full 60 covering being optional) as described above is placed in its collapsed state into the proximal end of the delivery catheter 30. The intraluminal device 1 can be placed into the delivery catheter 30 with either the first 20 or second 21 guide bar leading. The first 20 and second 21 guide bars allows the physician to insert the delivery catheter 30 into the body vessel 50 from either direction. For example, in FIG. 5A, the delivery catheter 30 is inserted into the body vessel 50 such that the pushing mechanism 35 makes contact with the first 20 guide bar (e.g., second 21 guide bar leading in the same direction of fluid flow). While in FIG. 5B, the delivery catheter 30 is inserted into the body vessel 50 such that the pushing mechanism 35 makes contact with the second 21 guide bar (e.g., the first 20 guide bar leading in opposite direction of fluid flow).

The pushing mechanism 35 is then inserted into the proximal end of the delivery catheter 30. The end of the pushing mechanism 35 is adapted to make contact with either the first 20 or second 21 guide bar and to push the intraluminal device 1 through the delivery catheter 30. The intraluminal device 1 exits the distal end of the catheter 30 for delivery to the targeted location in the body vessel 50. Finally, the intraluminal device 1 moves from the collapsed state to the expanded state for engagement with the wall 55 of the body vessel 50.

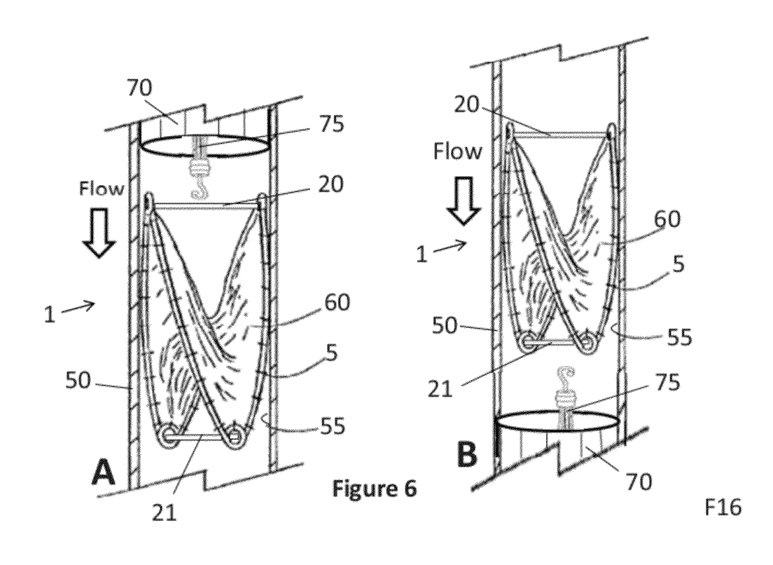

The method may also include steps through which the intraluminal device 1 is retrieved from the body vessel upon completion of its intended application or when desirable. Referring now to FIGS. 6A and 6B, retrieving the intraluminal device 1 from the targeted location in the body includes, first, introducing a retrieval catheter 70 having a proximal and distal end into the body vessel 50. The retrieval catheter 70 may be different from or the same as the delivery catheter 30. A retrieval hook 75 is inserted into the proximal end of the retrieval catheter 70 and pushed through to the catheter's distal end. The end of the retrieval hook 70 adapted to grasp either the first 20 or second 21 guide bar and to move or pull the intraluminal device 1 through the retrieval catheter 70. The end of the retrieval hook 70 may be any shape or configuration that allows the hook 70 to make contact with and grasp or couple to one of the guide bars 20, 21. Prior to entering the retrieval catheter 70, the intraluminal device 1 moves from its expanded state to its collapsed state, thereby, reducing contact with the wall 55 of the body vessel 50. Finally, the intraluminal device 1 is retrieved from the body vessel 50.

The first 20 and second 21 guide bars allow the physician to insert the retrieval catheter 70 into the body vessel 50 from either direction. For example, in FIG. 6A, the retrieval catheter 70 is inserted into the body vessel 50 such that the retrieval hook 75 makes contact with the first 20 guide bar (e.g., first 20 guide bar leading the retrieval in the opposite direction of fluid flow). While in FIG. 6B, the retrieval catheter 70 is inserted into the body vessel 50 such that the retrieval hook 75 makes contact with the second 21 guide bar (e.g., the second 20 guide bar leading the retrieval in the same direction of fluid flow).

A person skilled in the art will recognize that the measurements described are standard measurements that can be obtained by a variety of different test methods. The test methods described in the examples represents only one available method to obtain each of the required measurements.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. An intraluminal medical device, the intraluminal device comprising:
a self-expanding frame having a plurality of side elements interconnected by a plurality of oppositely facing bends; the frame configured to move between a collapsed state in which the bends are compressed into near proximity with the side elements for delivery and retrieval and an expanded state in which the frame expands allowing the side elements to engage the body vessel; and
a pair of guide bars located on opposites ends of the frame; the guide bars configured to couple with the oppositely facing bends such that the bars enable the frame to be retracted for delivery or retrieval from either end.

2. The intraluminal device of claim 1, wherein the frame is comprised of one selected from the group of stainless steel and a superelastic material.

3. The intraluminal device of claim 1, wherein the frame further comprises at least one attachment mechanism resulting in the frame having a closed circumference.

4. The intraluminal device of claim 3, wherein the frame is constructed of one selected from the group of a single component or multiple components.

5. The intraluminal device of claim 1, wherein the frame includes a surface treatment or coating of a therapeutic agent.

6. The intraluminal device of claim 3, wherein the frame has a polygonal shape.

7. The intraluminal device of claim 1, wherein the guide bars are adapted to flex or bend in order to allow the device to move from the expanded state to the collapsed state.

8. The intraluminal device of claim 1, wherein the guide bars are comprised of one selected from the group of a superelastic material and a flexible polymer matrix.

9. The intraluminal device of claim 1, wherein the device further comprises a wire, strut, or structure attached to at least one of the sides of the frame; the wire, strut, or structure having a first end and a second end;
wherein at least one of the first end or second end of the wire, strut, or structure includes a barb to anchor the device to the body vessel.

10. The intraluminal device of claim 9, wherein the barb is one selected from the group of a hook, a bend, or a sharpened point.

11. The intraluminal device of claim 3, wherein the device further comprises one selected from the group of a partial covering and a full covering; the partial and full coverings being attached to at least a portion of the circumference of the frame.

12. The intraluminal device of claim 11, wherein the full covering is attached to a substantial portion of the frame's circumference.

13. The intraluminal device of claim 11, wherein the partial covering is triangular in shape.

14. The intraluminal device of claim 11, wherein the covering is comprised of one selected from the group of a fabric, collagen, or a flexible material.

15. The intraluminal device of claim 11, wherein the covering is attached to the frame using one selected from the group of sutures, adhesive, heat sealing, weaving, and cross-linking.

16. A stent for use in maintaining an open lumen in a body vessel, the stent comprising the intraluminal device of claim 3.

17. An artificial valve for use in correcting valvular incompetence, the valve comprising the intraluminal device of claim 13.

18. An occlusion device for preventing the flow of fluid through a body vessel; the occlusion device comprising the intraluminal device of claim 12.

19. A method for delivering an intraluminal device to a targeted location in a body vessel for use as a stent, an occlusion device, or a valve; the method comprising the steps of:
introducing a delivery catheter having a proximal and distal end into the body vessel; the distal end being located proximate to a targeted location in the body vessel;
providing an intraluminal device; the intraluminal device comprising:
a self-expanding frame having a plurality of side elements interconnected by a plurality of oppositely facing bends; the frame configured to move between a collapsed state in which the bends are compressed into near proximity with the side elements for delivery and retrieval and an expanded state in which the frame expands allowing the side elements to engage the blood vessel; and
first and second guide bars located on opposites ends of the frame; the first and second guide bars configured to couple with the oppositely facing bends;
inserting the intraluminal device in the collapsed state into the proximal end of the delivery catheter;
inserting an end of a pushing mechanism into the proximal end of the delivery catheter; the end of the pushing mechanism adapted to make contact with one of the first or second guide bars; the pushing mechanism adapted to move the intraluminal device through the delivery catheter;
delivering the intraluminal device to the targeted location in the body vessel; and
allowing the intraluminal device to move to the expanded state for engagement with the body vessel.

20. The method of claim 19, wherein the method further comprises the steps of:
introducing a retrieval catheter having a proximal and distal end into the body vessel; the distal end being located proximate to the targeted location in the body vessel;
inserting an end of a retrieval mechanism into the proximal end of the retrieval catheter; the end of the retrieval mechanism adapted to grasp one of the first or second guide bars; the retrieval mechanism adapted to move the intraluminal device through the retrieval catheter;
allowing the intraluminal device to move from the expanded state to the collapsed state; and
retrieving the intraluminal device from the targeted location in the body vessel.

21. The method of claim 19, wherein the step of providing an intraluminal device uses an intraluminal device that further comprises a covering.
